(19) United States
(12) Patent Application Publication
(10) Pub. No.: US 2023/0143553 A1
(43) Pub. Date: May 11, 2023

(54) PRECISION DELIVERY OF MULTI-SCALE PAYLOADS TO TISSUE-SPECIFIC TARGETS IN PLANTS

(71) Applicant: MASSACHUSETTS INSTITUTE OF TECHNOLOGY, Cambridge, MA (US)

(72) Inventors: Benedetto MARELLI, Lexington, MA (US); Yunteng CAO, Cambridge, MA (US)

(21) Appl. No.: 17/907,603

(22) PCT Filed: Apr. 16, 2021
(86) PCT No.: PCT/US2021/027639

§ 371 Date: Sep. 28, 2022

Related U.S. Application Data
(60) Provisional application No. 63/011,622, filed on Apr. 17, 2020.

(51) Int. Cl. A01N 25/10; A01P 21/00; C12P 21/06; C12N 9/50

(52) U.S. Cl. CPC A01N 25/10; A01P 21/00; C12P 21/06; C12N 9/50; C12Y 304/21001

(57) ABSTRACT

The invention provides a silk-based biomaterial designed to fabricate a microneedle-like device with controlled degradation rate in planta via tuning material composition and methods of manufacture thereof. The device enables precise access to xylem, phloem, and other plant tissues, thus being capable of serving as delivery tool to release cargo payloads and as sampling tool for analysis of plants fluid.

### RELATED APPLICATION

This application claims the benefit of U.S. Provisional Application No. 63/011,622 filed Apr. 17, 2020. The entire teachings of the above-referenced application are incorporated herein by reference.

### BACKGROUND

A projected world population of 9.7 billion people in 2050 may result in a 70% increase of food demand and pose a severe strain to global food security.^{[1]} To address these challenges, innovations in plant genetic engineering and precision agriculture are highly sought to enhance crop productivity, impart and/or enhance plants resistances to diseases and stresses and increase the sustainability of crop production.^{[2]} In this scenario, there is an increasing interest in the use of biomaterials and nanotechnology to plant science and crop production, provided the tremendous effects of these technologies in biomedicine (e.g. drug delivery) and microbiology. For example, nanomaterials have been used in plants as bactericides and fertilizers,^{[3, 4]} microneedles have been applied on leaves to sample pathogenic bacteria^{[5]} and nanobionics has been developed to impart new function to plants' organelles.^{[6, 7]} Nonetheless, the use of biomaterials and drug delivery principles to engineer the precise deployment of payloads in plants has been largely overseen. This has also resulted in limited technical capability in dealing with diseases that target plant vasculature (e.g. phloem- or xylem-restricted bacterial^{[8]}) and is a limiting factor in plant engineering, where nanoparticles are delivered to plant tissues by complex and inefficient methods, such as foliar spray, gene guns, soil absorption, and syringe infiltration.

Silk fibroin (derived from Bombyx mori) has been extensively studied as technical material in a wide range of fields including drug delivery and regenerative medicine,^{[9]} optoelectronics,^{[10]} and food coatings^{[11]} due to mechanical robustness, tunable degradation in non-toxic byproducts, preservation of payloads, and ease of fabrication. These features are attractive also for the design of a plant-specific biomaterial for drug delivery. However, limited free water and low concentration of proteases in plant sap fluid result in prolonged silk fibroin stability and limited release of cargo molecules.^{[12]}

Therefore this is a need for new compositions and methods for delivering payloads to tissue-specific targets in plants.

### SUMMARY OF THE INVENTION

This Summary introduces a selection of concepts in simplified form that are described further below in the Detailed Description. This Summary neither identifies key or essential features, nor limits the scope, of the claimed subject matter.

In an embodiment, the invention provides a composition comprising a mixture of polypeptides derived from silk fibroin (Cs) that have a higher hydrophilic content than pure silk fibroin; and pure silk fibroin (SF).

In an embodiment, the invention provides a method for manufacturing a silk fibroin material having a higher hydrophilic content than pure silk fibroin. In embodiments, the method comprises contacting pure SF with a proteolytic enzyme under conditions sufficient to degrade SF proteins thereby forming hydrophilic polypeptides (Cs); and combining the Cs with SF to form a mixture having a higher hydrophilic content than pure SF.

In an embodiment, the invention provides a method for delivering a payload to a locus in plant tissue. In embodiments, the method comprises providing a ratio of Cs:SF material configured as a microneedle device having a higher hydrophilic content than pure SF; loading the microneedle device with the payload; and contacting the plant tissue with the microneedle device under conditions sufficient to allow the payload to enter the locus.

In an embodiment, the invention provides a microneedle comprising the composition of claim 1, wherein the microneedle comprises a base and at least one penetrating tip.

In an embodiment, the invention provides a microneedle comprising greater than 90% SF comprising a base and a penetrating tip, wherein the penetrating tip is capable of sampling fluid in the xylem or phloem of a plant. In embodiments, the microneedle comprises 100% SF.

In an embodiment, the invention provides a diagnostic kit for diagnosing the presence of a pathogen in the xylem or phloem of a plant.

The following Detailed Description references the accompanying drawings which form a part this application, and which show, by way of illustration, specific example implementations. Other implementations may be made without departing from the scope of the disclosure.

### BRIEF DESCRIPTION OF THE DRAWINGS

The patent or application file contains at least one drawing executed in color. Copies of this patent or patent application publication with color drawings will be provided to the Office upon request and payment of the necessary fee.

The foregoing and other objects, features and advantages of the invention will be apparent from the following more particular description of preferred embodiments of the invention, as illustrated in the accompanying drawings in which like reference characters refer to the same parts throughout the different views. The drawings are not necessarily to scale, emphasis instead being placed upon illustrating the principles of the invention.

FIG. 1A through FIG. 1F depicts material and device design for multiscale, multi-tissue precise delivery of payloads in plants. FIG. 1A: Material design: Silk materials were engineered to perform in plants. Silk fibroin is first extracted from Bombyx mori cocoons; the 390 kDa heavy chain is composed of 12 hydrophobic blocks (red cylinders) staggered by 11 hydrophilic spacers (blue lines). By using alpha-chymotrypsin, the hydrophilic spacers (Cs) can be extracted. The final material is a blend of Cs and silk fibroin, which is fabricated into phytoinjectors via molding. FIG. 1B: Silk fibroin materials can be fabricated in arrays of microneedles (here called phytoinjectors) of desired size and shape for precise payloads delivery in different plant tissues. In the schematic, injection in foliar tissue, shoot apical meristem and plant vasculature are represented. In particular, the green and red injectors indicate delivery to xylem and phloem, respectively. The left inset indicates delivery to shoot apical meristem (SAM). FIG. 1C, FIG. 1D, FIG. 1E, and FIG. 1F are scanning electron micrographs of phytoinjectors designed for delivery to SAM, leaf, xylem, and phloem, respectively. The inlets show the injectors' tips. Scale bar: 100 μm, scale bar of inlet: 20 μm.

FIG. 2A through FIG. 2F depict material characterization of engineered silk material for in planta applications. FIG. 2A shows the solubility of Cs-silk fibroin blends (CsₓₓSF_{yy}) in simulated sap fluid. Cs dramatically increases the solubility of CsSF blends, resulting in materials that can easily biodegrade in a sap-like environment. FIG. 2B depicts CD spectra of CsSF blends with various Cs content. FIG. 2C depicts the mechanical properties of CsSF films with various Cs content under tension. FIG. 2D depicts hydrogen peroxide preservation in SF, Cs₁₀SF₉₀, and Cs₂₀SF₈₀. FIG. 2E depicts HRP preservation in SF, Cs₁₀SF₉₀, and Cs₂₀SF₈₀. FIG. 2F depicts Agrobacterium preservation in SF and Cs₂₀SF₈₀. Data are mean±s.d (n is at least 3).

FIG. 3A through FIG. 3F depict payload delivery in stem's vasculature system. FIG. 3A depicts a tomato plant injected in the stem by an array of phytoinjectors loaded with rhodamine 6 g. The phytoinjector array is showed on top left. Scale bar, 1 mm. FIG. 3B shows a cross section of the injection site, depicting a phytoinjector that reaches tomato stem's vasculature system. Scale bar, 500 μm. FIG. 3C is a bright field image of a histological section of stem's cross section at injection site. Scale bar, 200 μm. FIG. 3D depicts fluorescent microscope images showing rhodamine 6 g delivered to and transported in phloem, from source to sink. The red spots highlighted by white arrows point to rhodamine 6 g in phloem. Scale bar, 500 μm. FIG. 3E depicts image assembly of fluorescent micrographs showing 5(6)-carboxyfluorescein diacetate delivered to and transported in xylem, from roots to canopy, 1, 3, and 5 minutes post injection. FIG. 3F shows corresponding fluorescent intensity depicting 5(6)-carboxyfluorescein diacetate distribution along xylem (1, 3, and 5 minutes post injection, respectively). Red dash line highlights the saturated zone due to residue of the phytoinjetor, which is removed from experimental data. Black dot line is the background. Solid curves are experimental data while dash dot lines with the same color are corresponding model simulation.

FIG. 4A through FIG. 4F depict delivery and sampling of biomolecules in xylem. FIG. 4A depicts delivery of luciferin into the petiole xylem; by providing external luciferase, ATP, and Mg²⁺, the whole leaf emits light. (Exposure time 30 seconds, image adjusted for display purpose) FIG. 4B depicts two arrays of phytoinjectors loaded with different payloads (luciferin for blue injectors and luciferase for red ones, blue and red here are only for display purpose) targeting petiole's xylem concurrently. By providing external ATP and Mg²⁺, the leaf vein emits light (Exposure time 120 seconds, image adjusted for display purpose). FIG. 4C shows sampling of luciferin and Mg′ delivered to petiole xylem by Cs₂₀SF₈₀ phytoinjectors using an SF phytosampler (Exposure time 30 seconds for dark field). FIG. 4D depicts swelling of and water movement in a phytosampler injected into agar gel. indicating the possible use to sample plant fluids. FIG. 4E shows corresponding water penetration length with time. FIG. 4F depicts a phytosampler injected into toluidine blue agar gel becomes blue in 1 minute. Data are mean±s.d (n=3).

FIG. 5A through FIG. 5C depict Agrobacterium-mediated gene transfer to shoot apical meristem and leaves. FIG. 5A(i) through FIG. 5A(iii) depict Agrobacterium delivered to the shoot apical meristem. (i) shoot apical meristem (SAM) injected by a phytoinjector loaded with agrobacteria (rhodamine 6 g was also loaded for display purpose); (ii) bright and dark field images of the leaf from the shoot 2 weeks after the injection. Bright green spots in dark field indicating GFP expressed in leaf cells are distributed across the whole leaf; and (iii), fluorescent microscope images of the leaf in ii. Agrobacterium delivered to a young leaf FIG. 5B(i) through FIG. 5B(iii) and to a mature leaf FIG. 5C(i) through FIG. 5C(iii): i, ii, and iii are images when injected, bright and dark field images 2 weeks after injection, and fluorescent microscope images of the injected area on leaves. GFP is observed away from the injection site in a young leaf due to tissue growth, while it expressed only at the injection site in a mature leaf. Scale bar 2 mm for i and ii, 500 μm for iii. Exposure time, bright field 20 ms, dark field 5 seconds.

FIG. 6A through FIG. 6C. Cs fabrication and material size distribution. a, photographs of silk fibroin solution, gel formed after 24 h incubation at 37° C. by silk fibroin and alpha-chymotrypsin, and Cs after centrifuge. b, SDS-PAGE of silk fibroin(SF), Cs, Cs₂₀SF₈₀, and GST tagged GFP (˜53 kDa). c, Size distribution of as prepared Cs₂₀SF₈₀, SF and resuspended Cs₂₀SF₈₀ and SF. Pure Cs solution has a hydrodynamic radius below 1 nm.

FIG. 7A through FIG. 7C. ATR-FTIR spectra of CsSF blend and quantification of secondary structure. a, ATR-FTIR spectra of CsSF blend with increasing Cs content. All the investigated ratios of silk:Cs showed similar spectra with a strong peak at 1645 cm⁻¹ indicating water-soluble random coil conformation. b, Self-deconvolution curve of the ATR-FTIR spectrum of SF and peak fitting. Black solid line is the self-deconvoluted curve, red dot line is the fitted curve by individual peaks (green). c. Percentage of secondary structures in CsSF blends with increasing Cs content. Error bar means s.d.

FIG. 8 Raman spectra of Cs, Cs₂₀SF₈₀, and SF. Solid lines indicates samples that are as prepared while dotted lines refer to samples treated in 80% v/v methanol. Cs shows a polymorphic behavior upon exposure to methanol as it undergoes a random coil to beta-sheet transition (changes in Amide I and III peaks).

FIG. 9A and FIG. 9B. Thermogravimetric analysis (TGA) and differential scanning calorimetry (DSC) thermograms of Cs, Cs₂₀SF₈₀, and SF.

FIG. 10. Raman spectra of Cs₂₀SF₈₀ with and without H₂O₂. The characteristic band of H₂O₂ 880 cm⁻¹ shifts to 869 cm⁻¹ due to the contribution of a protein band at 852 cm⁻¹.

FIG. 11A through FIG. 11C. Mechanical properties of CsSF blend. a, Stress-strain curves of CsSF blends. b, Force-displacement curves of nanoindentation of CsSF blends. c, Reduced Young's modulus of CsSF blends. Error bar means s.d.

FIG. 12A through FIG. 12D. Release of payload models in simulated sap. a, Rhodamine 6 g (left), azoalbumin (middle), and R. tropici (right) preserved in SF and Cs₂₀SF₈₀ release in simulated sap. All the payloads encapsulated in the two materials follow a power law release. Cs₂₀SF₈₀ showed an increased release rate than SF. b, Scanning electron micrographs of Cs₂₀SF₈₀ with different payloads. The surfaces of rhodamine 6 g and azoalbumin loaded are flat and smooth, while the surface of R. tropici loaded materials shows the bacteria profiles. c and d, SEM images of SF and Cs₂₀SF₈₀ materials after 5 mins exposure to simulated sap. Scale bar, 10 μm. Data are mean±s.d (n=3).

FIG. 13A through FIG. 13D. Phytoinjectors targeting on xylem and phloem of tomato plants. a, Tomato petiole cross section. Phloem (deep green) and xylem (pink) are regularly arranged. Scale bar 500 μm for the left and 50 μm for the right. b, Depth of phloem and xylem in tomato petiole. c and d, photograph of phytoinjectors for xylem and phloem, respectively. Scale bar 1 mm.

FIG. 14A through FIG. 14D. Mechanical behavior of phytoinjectors and plant tissues during injection with a xylem phytoinjector. a, Mechanical behavior of xylem and phloem phytoinjectors fabricated from Cs₂₀SF₈₀ and SF under compression. The phytoinjectors mainly break due to bending because the inevitable lateral force exerted during compression. The phloem phytoinjector fabricated from Cs₂₀SF₈₀ may undergo material cracking as the force was maintained around 0.1 N where the displacement is from 15 μm to 25 μm (The tip of a phloem injector is <10 μm in diameter). Reaction forces during injection of a xylem phytoinjector into tomato (b), tobacco (c), and citrus (d). Dotted lines represent the completion of the injection, where the whole phytoinjector was inside the tissue plant or the tissue (leaf) was injected through.

FIG. 15A through FIG. 15C. Stele types and wound caused by phytoinjectors a, Different types of steles. b, Leaf cell viability post injection. Cells stained blue by toluidine blue are dead while not stained are alive. Scale bar 100 μm. c, Wound on tomato petiole caused by xylem phytoinjectors, immediate, 1, 3, 7, and 14 days post injection. Scale bar 1 mm.

FIG. 16A through FIG. 16E. Standard curves. a. rhodamine 6G, b. azozlbumin, c. R. tropici, d. H₂O₂, and e. HRP, respectively. R² here is adjusted adjusted R-squared. Error bar means s.d.

FIG. 17. Schematic of the model.

### DETAILED DESCRIPTION

To overcome these challenges in the with delivering payloads to tissue-specific targets in plants, the present invention provides a new biomaterial based on silk fibroin that was formatted in a device capable of delivering a variety of payloads ranging from small molecules to large proteins into specific loci of various plant tissues.

In embodiments, the invention provides a composition comprising a mixture of polypeptides derived from silk fibroin (Cs) that have a higher hydrophilic content than pure silk fibroin; and pure silk fibroin (SF).

In embodiments, the dry weight ratio of Cs:SF in the composition is between 10:90 and 40:60. In embodiments, the dry weight ratio of Cs:SF in the composition is between 10:90 and 30:70. In embodiments, the dry weight ratio of Cs:SF in the composition is between 10:90 and 20:80. In embodiments, the dry weight ratio of Cs:SF in the composition is 20:80.

In embodiments, the dry weight percentage of Cs in the composition is at least 21%. In embodiments, the dry weight percentage of Cs in the composition is at least 40%.

In embodiments, the invention provides a microneedle comprising a mixture of polypeptides derived from silk fibroin (Cs) that have a higher hydrophilic content than pure silk fibroin; and pure silk fibroin (SF); wherein the microneedle comprises a base and at least one penetrating tip.

In embodiments, the dry weight ratio of Cs:SF in the composition is between 10:90 and 40:60. In embodiments, the dry weight ratio of Cs:SF in the composition is between 10:90 and 30:70. In embodiments, the ratio of Cs/SF in the microneedle is between 10:90 and 20:80. In embodiments, the dry weight ratio of Cs:SF in the composition is 20:80.

In embodiments, the dry weight percentage of Cs in the composition is at least 21%. In embodiments, the dry weight percentage of Cs in the composition is at least 40%.

In embodiments, the penetrating tip length of the microneedle is suitable for penetration of the microneedle to the xylem of phloem of a plant. In embodiments, the penetrating tip diameter of the microneedle is capable of penetrating the xylem or phloem of a plant without disrupting the flow of material in the xylem or phloem.

In embodiments, the invention provides a microneedle comprising a mixture of polypeptides derived from silk fibroin (Cs) that have a higher hydrophilic content than pure silk fibroin; and pure silk fibroin (SF); wherein the microneedle has a base and a penetrating tip, wherein the microneedle comprises greater than 90% SF, and wherein the penetrating tip is capable of sampling fluid in the xylem or phloem of a plant.

In embodiments, the invention provides a microneedle comprising 100% SF wherein the microneedle has a base and a penetrating tip, and wherein the penetrating tip is capable of sampling fluid in the xylem or phloem of a plant.

The microneedle's mechanical robustness and solubility was controlled by tuning the relative amount of hydrophobic/hydrophilic domains, which enabled the design and fabrication of an array of injectors (referred to herein as phytoinjector) capable of targeting plant vasculature by penetrating plant dermal and ground tissues. The dimensions of tissue-specific phytoinjectors were determined by histological analysis of the target tissue. Using specific phytoinjectors, payloads (ranging in size from small molecules to large proteins) were deployed in tomato plant xylem and phloem and their transport to sink and source was observed and modeled. Agrobacterium-loaded phytoinjectors also showed gene transfer to and expression in tobacco shoot apical meristem (SAM) and in leaves at various stages of growth. Tuning of material composition also enabled the fabrication of a device to sample xylem sap.

In embodiments, the invention provides a method for manufacturing a silk fibroin material having a higher hydrophilic content than pure silk fibroin (SF). In embodiments, the method comprises contacting pure SF with a proteolytic enzyme under conditions sufficient to degrade SF proteins thereby forming hydrophilic polypeptides (Cs); and combining the Cs with SF to form a mixture having a higher hydrophilic content than pure SF.

In embodiments, the dry weight ratio of Cs:SF in the composition is between 10:90 and 40:60. In embodiments, the dry weight ratio of Cs:SF in the composition is between 10:90 and 30:70. In embodiments, the ratio of Cs/SF in the microneedle is between 10:90 and 20:80. In embodiments, the dry weight ratio of Cs:SF in the composition is 20:80.

In embodiments, the dry weight percentage of Cs in the composition is at least 21%. In embodiments, the dry weight percentage of Cs in the composition is at least 40%.

In some embodiments, the microneedle comprises greater than 90% SF.

In embodiments, the invention provides a method for delivering a payload to a locus in plant tissue. In embodiments, the method comprises providing a ratio of Cs:SF material configured as a microneedle device having a higher hydrophilic content than pure SF; loading the microneedle device with the payload; and contacting the plant tissue with the microneedle device under conditions sufficient to allow the payload to enter the locus.

In embodiments, the dry weight ratio of Cs:SF in the composition is between 10:90 and 40:60. In embodiments, the dry weight ratio of Cs:SF in the composition is between 10:90 and 30:70. In embodiments, the ratio of Cs/SF in the microneedle is between 10:90 and 20:80. In embodiments, the dry weight ratio of Cs:SF in the composition is 20:80.

In embodiments, the dry weight percentage of Cs in the composition is at least 21%. In embodiments, the dry weight percentage of Cs in the composition is at least 40%.

As used in any embodiment herein, the term “silk fibroin” includes silkworm fibroin and insect or spider silk protein. See e.g., Lucas et al., 13 Adv. Protein Chem. 107 (1958). Any type of silk fibroin may be used according to aspects of the present invention. Silk fibroin produced by silkworms, such as Bombyx mori, is the most common and represents an earth-friendly, renewable resource.

As used in any embodiment herein, the term “penetrating tip” refers to an end of a microneedle that is adapted to first contact and penetrate a surface, e.g., of a biological barrier. The penetrating tip can be of any shape and/or dimension. The penetrating tip can have a shape of various geometries, e.g., but not limited to, circles, rectangles, squares, triangles, polygons, and irregular shapes. In some embodiments, the penetrating tip can appear as a point, for example, due to limited resolution of optical instruments, e.g., microscopes, and/or of human eyes. In some embodiments, the shape of the penetrating tip can be the same as or different from that of the cross section of the microneedle body.

As used in any embodiment herein, the term “dimension” as used herein generally refers to a measurement of size in the plane of an object. With respect to a penetrating tip of the microneedles described herein, the dimension of a penetrating tip can be indicated by the widest measurement of the shape of the penetrating tip. For example, the dimension of a circular tip can be indicated by the diameter of the circular tip. In accordance with the invention, the penetrating tip can have a dimension (e.g., a diameter) ranging from about 50 nm to about 50 μm, including from about 100 nm to about 40 μm, from about 200 nm to about 40 μm, from about 300 nm to about 30 μm, from about 500 nm to about 10 μm, or from about 1 μm to about 10 μm. In some embodiments, the penetrating tip can have a dimension (e.g., a diameter) of less than 35 μm. In some embodiments, the penetrating tip can have a dimension (e.g., a diameter) of less than 10 μm.

The base of the microneedles described herein is generally the opposite end of the penetrating tip. The base of the microneedles can be attached or secured to a solid substrate or a device for facilitating the penetration of the microneedles into a biological barrier. The base of the microneedle can be of any size and/or shape. The base can have a shape of various geometries, e.g., but not limited to, circles, rectangles, squares, triangles, polygons, and irregular shapes. In various embodiments, the shape of the base can follow that of the cross section of the microneedle body.

In some embodiments, the base dimension (e.g., a diameter) of the microneedles can range from 50 nm to about 1500 μm, from about 50 nm to about 1000 μm, from about 100 nm to about 750 μm, from about 250 nm to about 500 μm, or from about 500 nm to about 500 μm.

The microneedles described herein can be in any elongated shape suitable for use in delivering payloads to plants. For example, without limitations, the microneedle can be substantially cylindrical, wedge-shaped, cone-shaped, pyramid-shaped, irregular-shaped or any combinations thereof.

The shape and/or area of the cross section of the microneedles described herein can be uniform and/or vary along the length of the microneedle body. The cross-sectional shape of the microneedles can take a variety of shapes, including, but not limited to, rectangular, square, oval, circular, diamond, triangular, elliptical, polygonal, U-shaped, or star-shaped. In some embodiments, the cross section of the microneedles can have a uniform shape and area along the length of the microneedle body. In some embodiments, the cross section of the microneedles can have the same shape, with a varying area along the length of the microneedle body. In some embodiments where the microneedles are irregular-shaped, their cross sections can vary in both shape and area along the length of the microneedle body, or their cross sections can vary in shape (with a constant area) along the length of the microneedle body. In one embodiment, the microneedles described herein comprise a tapered body with a substantially circular cross section along the length of the microneedle body. The cross-sectional dimensions of the microneedle body can range from 50 nm to about 1500 μm, from about 50 nm to about 1000 μm, from about 100 nm to about 750 μm, from about 250 nm to about 500 μm, or from about 500 nm to about 500 μm.

The length of the microneedle body can vary from micrometers to centimeters, depending on a number of factors, e.g., but not limited to, types of tissue targeted for administration, required penetration depths, lengths of the uninserted portion of a microneedle, and methods of applying microneedles across or into a biological barrier. Accordingly, the length of the microneedle body can be selected and constructed for each particular application. In some embodiments, the length of the microneedle body can further comprise an uninserted portion, i.e. a portion of the microneedle that is not generally involved in tissue penetration. In those embodiments, the length of the microneedle body can comprise an insertion length (a portion of a microneedle that can penetrate into or across a biological barrier) and an uninserted length. The uninserted length can depend on applications and/or particular device designs and configurations (e.g., a microneedle adaptor or a syringe that holds a microneedle).

In some embodiments, the microneedles of the present invention can comprise at least one payload. As used in any embodiment herein, the term payload refers to any active agent providing the desired activity, response or reaction. The amount of payload distributed in the microneedles described herein can vary from picogram levels to milligram levels, depending on the size of microneedles and/or encapsulation efficiency.

In embodiments, the payload can be selected from at least one active agent selected from the group consisting of proteins, peptides, antigens, immunogens, vaccines, antibodies or portions thereof, antibody-like molecules, enzymes, nucleic acids, siRNA, shRNA, aptamers, viruses, bacteria, small molecules, cells, hormones, antibiotics, therapeutic agents, diagnostic agents, and any combinations thereof.

In some embodiments, non-limiting examples of active agents include organic materials such as horseradish peroxidase, phenolsulfonphthalein, nucleotides, nucleic acids (e.g., oligonucleotides, polynucleotides, siRNA, shRNA), aptamers, antibodies or portions thereof (e.g., antibody-like molecules), hormones (e.g., insulin, testosterone), growth factors, enzymes (e.g., peroxidase, lipase, amylase, organophosphate dehydrogenase, ligases, restriction endonucleases, ribonucleases, RNA or DNA polymerases, glucose oxidase, lactase), bacteria or viruses, other proteins or peptides, small molecules (e.g., drugs, dyes, amino acids, vitamins, antioxidants), lipids, carbohydrates, chromophores, light emitting organic compounds (such as luciferin, carotenes) and light emitting inorganic compounds (e.g., chemical dyes and/or contrast enhancing agents such as indocyanine green), immunogenic substances such as vaccines, antibiotics, antifungal agents, antiviral agents, therapeutic agents, diagnostic agents or pro-drugs, analogs or combinations of any of the foregoing. See, e.g., WO 2011/006133, Bioengineered Silk Protein-Based Nucleic Acid Delivery Systems; WO 2010/141133, Silk Fibroin Systems for Antibiotic Delivery; WO 2009/140588, Silk Polymer-Based Adenosine Release: Therapeutic Potential for Epilepsy; WO 2008/118133, Silk Microspheres for Encapsulation & Controlled Release; WO 2005/123114, Silk-Based Drug Delivery System; U.S. 61/477,737, Compositions and Methods for Stabilization of Active Agents, the contents of which are incorporated herein by reference in their entirety.

Another aspect provided herein is a microneedle device comprising a substrate and one or more microneedles described herein, wherein the microneedles are integrated or attached to the substrate and extend from the substrate; and each microneedle comprises a base and a penetrating tip. In some embodiments, the microneedle device can comprise a substrate and a microneedle. In some embodiments, the microneedle device can comprise a substrate and at least 2, at least 3, at least 4, at least 5, at least 6, at least 7, at least 8, at least 9, at least 10, at least 15, at least 20, at least 25, at least 30, at least 40, at least 50, at least 60, at least 70, at least 80, at least 90, at least 100 or more microneedles.

The base of the microneedle can be mounted to the substrate or formed as part of the substrate that can be rigid or flexible, for example, in the form of a film to conform to a surface. In some embodiments, a microneedle device of the present invention can include a substrate and one or more microneedles projecting from the substrate, preferably by a predefined distance.

In some embodiments, each microneedle can extend from the substrate to the same distance or to a different distance, thus a predefined profile of constant or varying microneedle depth penetrations can be provided in a single device. The length of each microneedle body can be selected to position the penetrating tip at a predefined distance from the base to provide penetration to a predefined depth for delivery of at least one active agent.

A plurality of microneedles can be arranged in a random or predefined pattern, such as an array. The distance between the microneedles and the arrangement of the plurality of microneedles can be selected according to the desired mode of and characteristics of the delivery. The microneedles can be biodegradable, bioerodible or otherwise designed to leave at least a portion of the microneedle in the tissue penetrated.

Each microneedle present on the microneedle device need not have the same microneedle length. In some embodiments, each microneedle on the microneedle device can have the same microneedle body length. In alternative embodiments, the microneedles on the microneedle device can have different microneedle body lengths. Thus, a predefined profile of constant or varying microneedle depth penetrations can be provided in a single microneedle device. In some embodiments, the body length of each microneedle can be tuned to adjust for the curvature of a surface.

The substrate of the microneedle device can be constructed from a variety of materials, including metals, ceramics, semiconductors, organics, polymers, and any composites thereof.

In embodiments, the invention provides a diagnostic kit for diagnosing the presence of a pathogen in the xylem or phloem of a plant comprising a microneedle of any embodiment described herein.

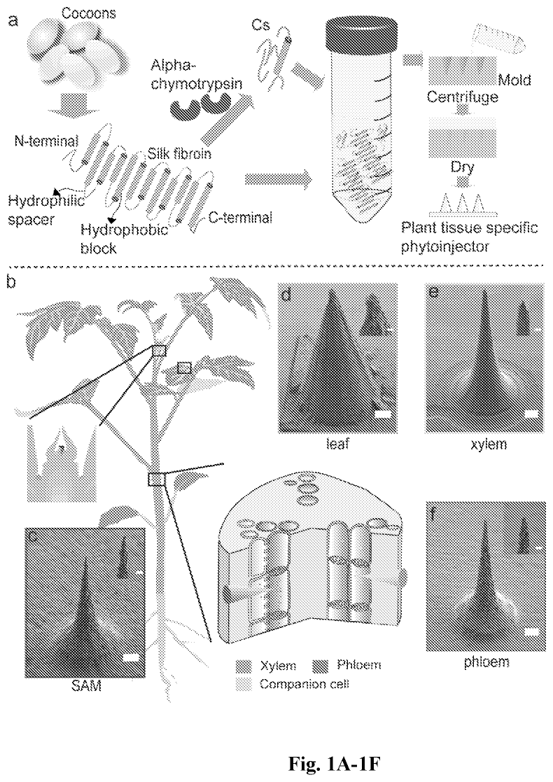

Preliminary investigations using silk fibroin showed limited payload release in xylem and phloem saps as well as flow interruption. To overcome these challenges, the invention provides a top-down synthetic approach to increase the hydrophilic content of the silk end-material (FIG. 1A, FIG. 6A) and decrease the size of the protein biodegradation byproducts by extracting hydrophilic silk fibroin-derived polypeptides (Cs) (FIG. 6B).^{[13]} Cs were then mixed with fresh silk fibroin water suspension to modulate fundamental biomaterial end-properties such as solubility, degradation, mechanical strength, nanomicelle size, and preservation of payloads. Materials characterization was accomplished to identify the optimal composition for payload delivery into plants. As used herein, a Cs 20%—Silk Fibroin 80% dry weight mixture is denoted as Cs₂₀SF₈₀.

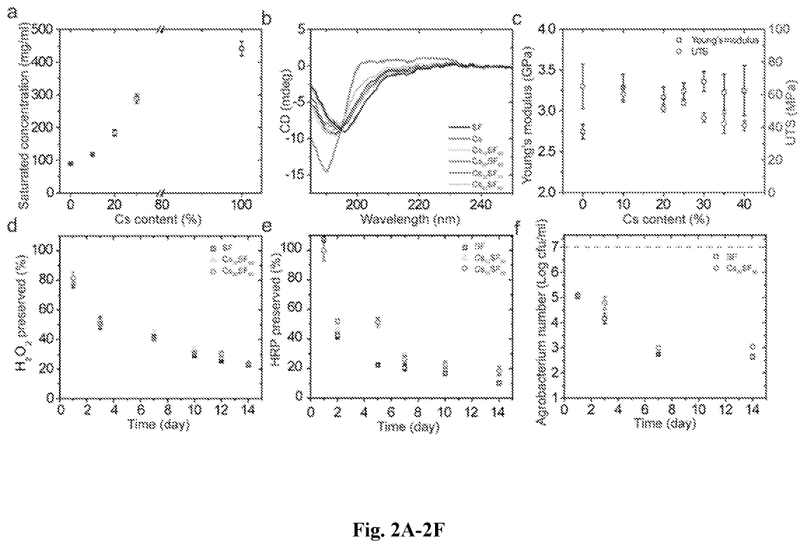

Cs-silk fibroin biomaterials were characterized according to the following properties: solubility, nanomicelle size when re-solubilized, conformation, viability of preserved labile payloads, and mechanical robustness. Solubility in simulated sap increases dramatically with increased Cs content (FIG. 2A). Compared to silk fibroin (89.8 mg ml⁻¹), the maximum concentration of Cs₂₀SF₈₀ in suspension is two times higher (184.1 mg ml⁻¹), while the concentration of pure Cs at saturation is five times higher (441.3 mg ml⁻¹). Nanomicelle size of resuspended Cs₂₀SF₈₀ has no significant difference from that of resuspended silk fibroin (FIG. 6C). The protein structure was investigated both in suspension by circular dichroism (CD) and in dry state (film form) by Attenuated Total Reflectance Fourier Transform Infrared Spectroscopy (ATR-FTIR). CD spectra of silk fibroin show a strong negative peak at 196 nm, indicating large amounts of random coils and a weak negative peak at 216 nm, distinctive of limited amounts of β-sheets.^{[14]}

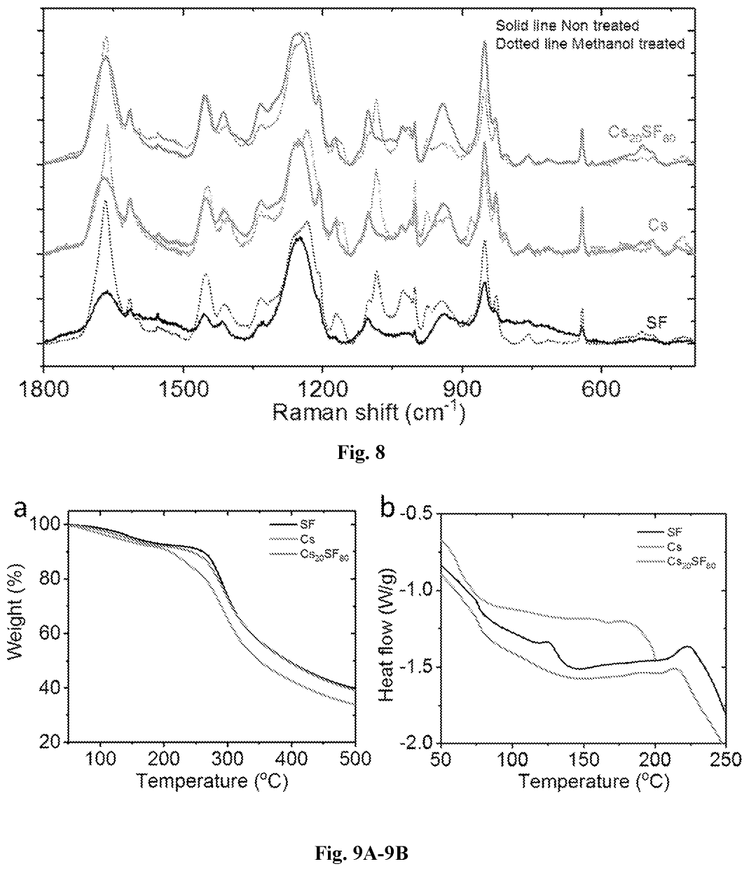

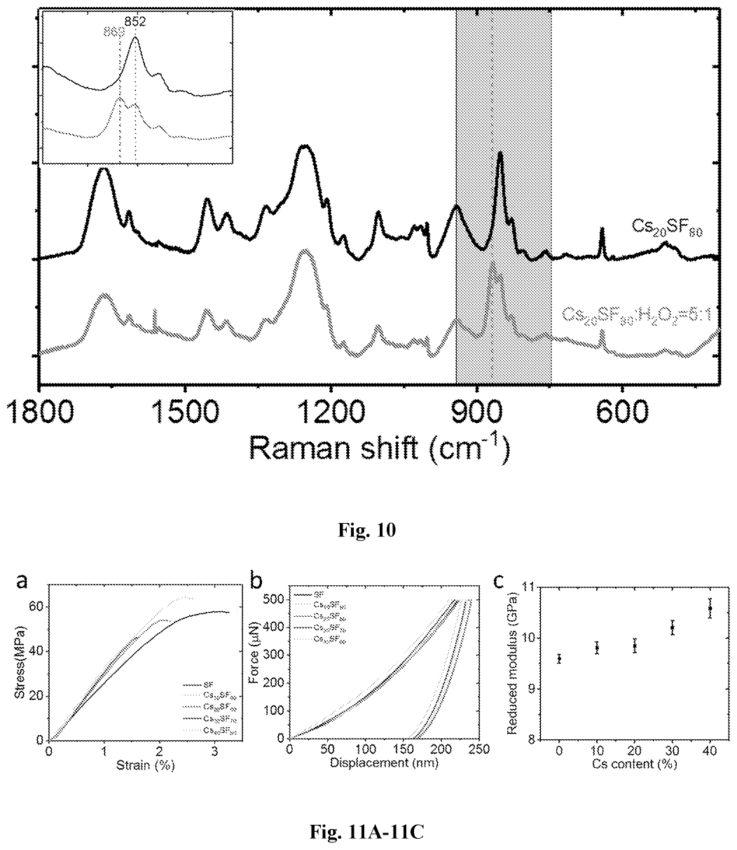

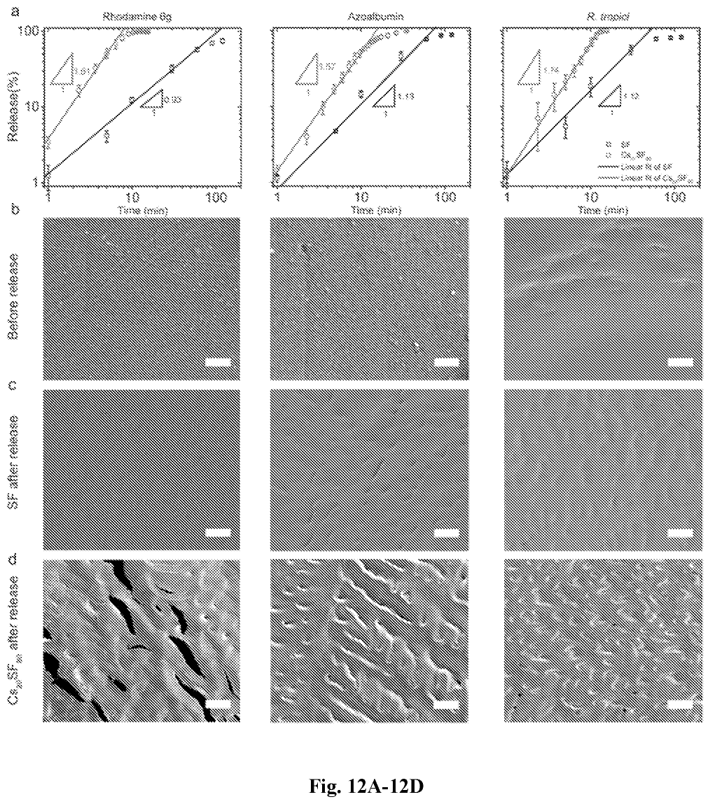

Pure Cs shows a strong negative peak at 190 nm and a weak negative peak at 216 nm, indicating the presence of β-turns and β-sheets, respectively (FIG. 2B). No noticeable conformation changes occur due to the blending of Cs and silk fibroin. FTIR spectra also show little difference and Amide I absorbance is dominated by a resonance centered at 1645 cm′ (FIG. 7A) that is characteristic of random coils.^{[15]} Incorporation of increasing concentrations of Cs in the blends did not result in a change of beta sheet content (FIG. 7B and FIG. 7C), suggesting that Cs did not drive a random coil to betasheet transition during silk fibroin assembly. The slight increase of turns with Cs content increase may attribute to the intrinsic molecule properties of Cs. Analysis of the Amide bands in Raman Spectra (FIG. 8) indicates that Cs does not hinder polymorphic changes of the structural protein. The difference of decomposition temperature of Cs (180° C.), SF (225° C.), and Cs₂₀SF₈₀ (205° C.) (FIG. 9A) indicates weakened molecular interaction between silk fibroin by Cs, which agrees with DSC results (FIG. 9B). Hydrogen peroxide was selected as a small molecule used for labile payload preservation due to its significant metabolic functions, which include lignification, ABA signaling in guard cells, programmed cell death and pathogen response.^{[16]} Based on the mechanical properties of the CsSF blends (FIG. 2C, discussed later), Cs content was limited to 20% or less. Hydrogen peroxide was well preserved in SF, Cs₁₀SF₉₀, and Cs₂₀SF₈₀, showing no significant differences among the three materials (FIG. 2D). In Cs₂₀SF₈₀ films, 81% and 50% of entrapped hydrogen peroxide was preserved at day 1 and 3 post-drying, respectively. At day 14, 24% of hydrogen peroxide was preserved, and this preservation window can be extended beyond three weeks. This strong oxidant is trapped in the Cs-silk fibroin matrix without chemical reactions, similarly to the presence of free water in the material (FIG. 10). Horseradish peroxidase (HRP) was used as a model to test preservation of enzymes and proteins. In this case, Cs₂₀SF₈₀ blends enhanced the preservation of the enzyme, which had 51% and 19% residual catalytic activity at days 5 and 14, respectively. To assess preservation of bacteria in Cs-silk fibroin blends, Agrobacterium tumefaciens was added to SF and Cs₂₀SF₈₀. The number of live bacteria preserved in dried SF and Cs₂₀SF₈₀ showed a 2-log reduction after 24 h, due to the drying process. At day 7, a further 2-logs decrease in bacteria viability was measured. Cs₂₀SF₈₀ shows a slightly improved performance in preserving Agrobacterium than SF (FIG. 2F). The feasibility of injecting CsSF mixtures in plants was first explored by investigating their mechanical properties via uniaxial tensile strength and nanoindentation measurements (FIG. 11A through FIG. 11C). SF Young's modulus was 2.75±0.09 GPa (FIG. 2C), which is in the range of previously reported measurements.^{[17]} The addition of Cs into silk fibroin materials enhances the Young's modulus by more than 15% but at the cost of ductility, which further confirms our proposed mechanism of interaction between Cs and silk fibroin. Nanoindentation results also indicate that reduced modulus increases with increasing Cs content from 0% up to 40%.

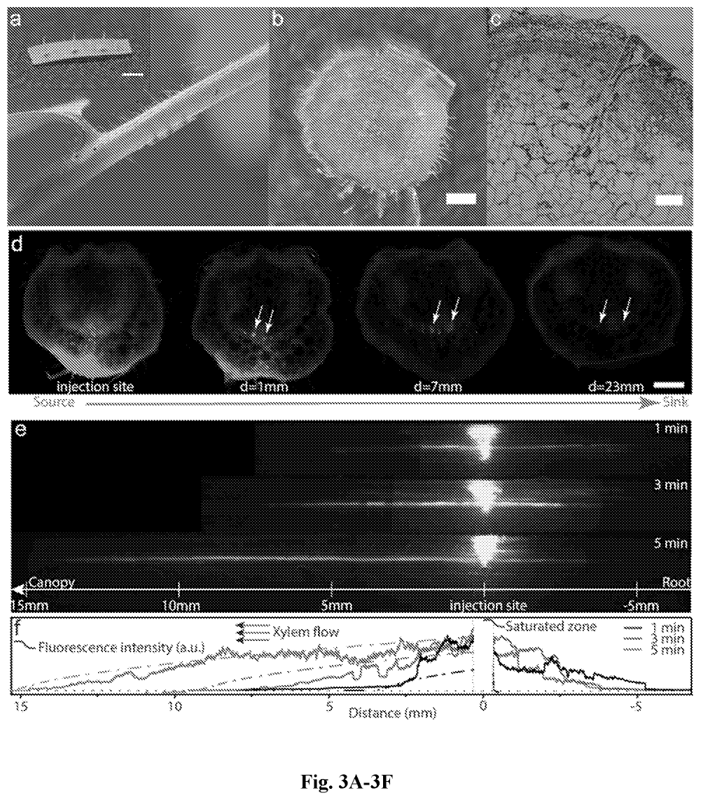

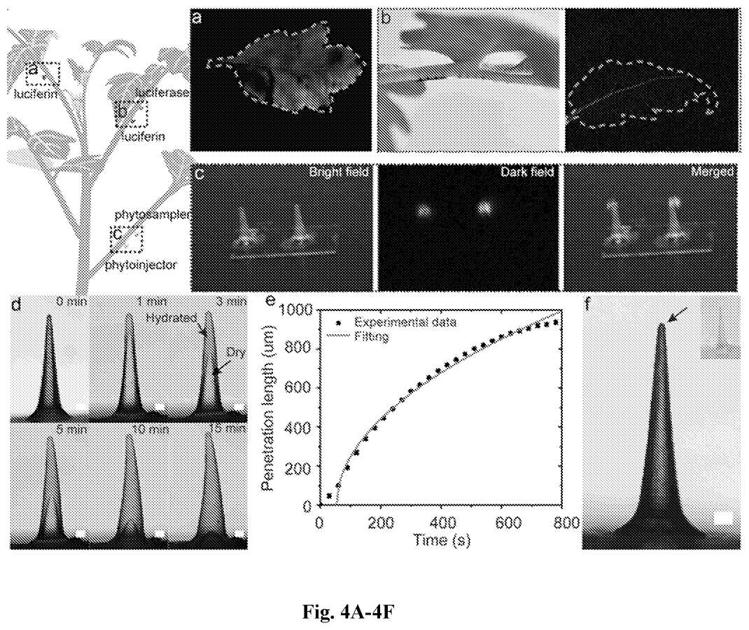

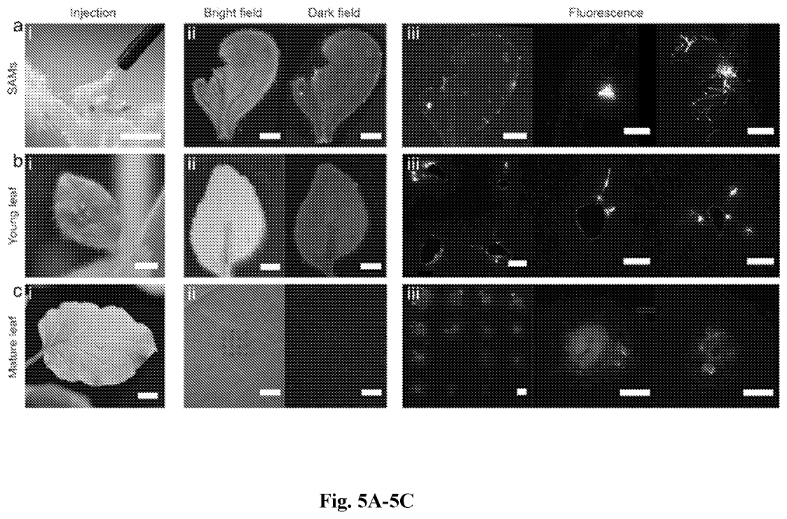

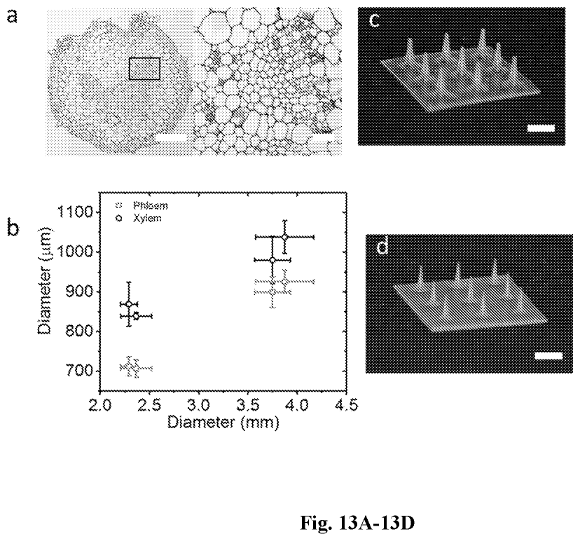

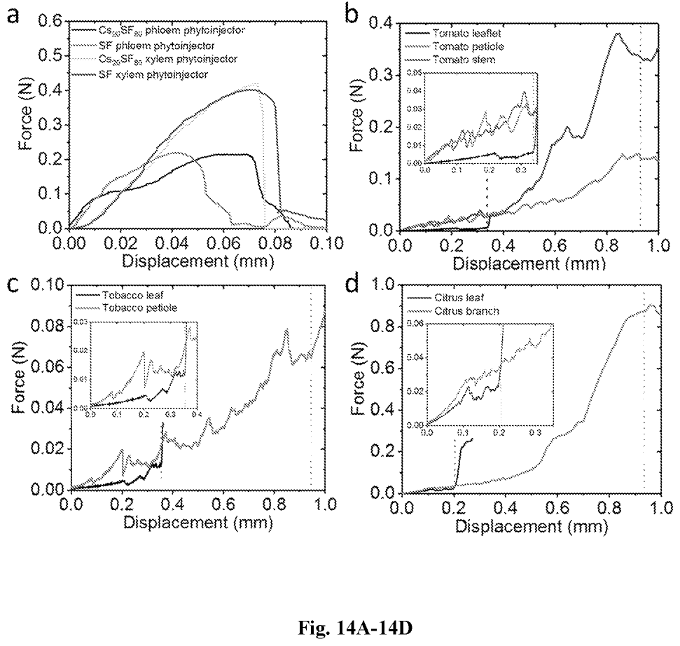

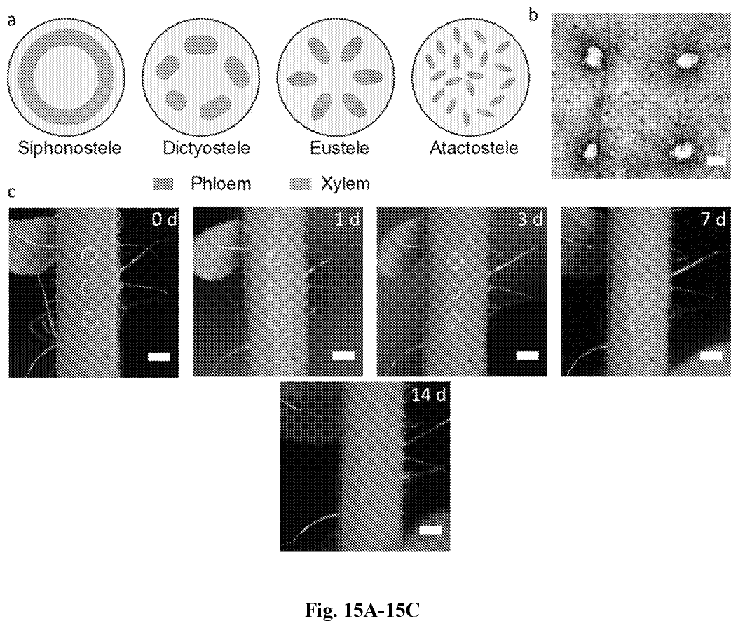

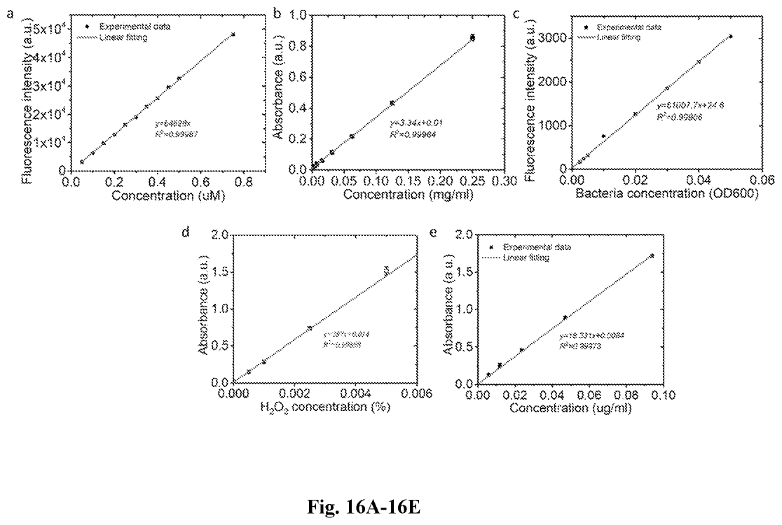

Payload release profiles of silk fibroin constructs in sap fluid follows a Super Case II mechanism (see Examples). To demonstrate targeted payload delivery to xylem and phloem, Cs₂₀SF₈₀ was combined with replica-molding to fabricate phytoinjectors of different sizes. To identify potential modes of entry to plant vasculature, histological samples of tomato (Solanum lycopersicum L.) stem and petiole were prepared and analyzed. Tomato was used as the working model because of the well-defined structure of the vasculature, presence of compound leaves with long petiole (FIG. 13A), and importance as crop. The penetration depth, defined as the segment between the vasculature and the epidermis, is in the range of 840-1040 μm and 707-925 μm for xylem and phloem, respectively, and depends on the diameter of petiole (FIG. 13B). The reported diameters of xylem and phloem are of the order of tens and hundreds of μm, respectively.^{[18]} Based on these parameters, phytoinjectors were designed with a tip diameter smaller than 35 μm and 10 μm for xylem and phloem, respectively (FIG. 1B, FIG. 13C and FIG. 13D). Resuspended Cs₂₀SF₈₀ has a particle size of 3-7 nm (FIG. 6C), which suggests that it can be transported in xylem through the pit membrane (pore size 5-420 nm^{[19]}) and in phloem through the sieve plate (pore size 610±150 nm in S. lycopersicum^{[20]}). Phytoinjectors exhibit appropriate mechanical robustness to for injection to various tissues of tomato plant, tobacco plant and citrus tree (FIG. 14). To investigate payload delivery in planta, each payload was loaded to phytoinjectors at the point of material assembly before drying. Rhodamine 6G and 5(6)-carboxyfluorescein diacetate were incorporated into phytoinjectors to target phloem and xylem, respectively, and injected in tomatoes' petioles (FIG. 3A). Petiole cross-section showed that the phytoinjectors reached the vasculature (FIG. 3B). Histological analysis also corroborated these findings (FIG. 3C). The injected petioles were sliced along the transverse section downstream and upstream at various distances from the injection site to investigate the presence of the delivered dyes. For phloem injections, rhodamine 6G was transported further downstream (i.e. from leaf to root for a mature leaf, >3.3 cm) than upstream (˜0.3 cm) the injection site. This result is in accordance with reported translocation in phloem for mature leaves^{[18, 21]} (FIG. 3D) and indicates that phytoinjectors successfully deployed payloads in the phloem that were translocated along the vascular tissue. In xylem, transport analysis was conducted by tangential sectioning of the stem (FIG. 3E). Analysis of 5(6)-carboxyfluorescein diacetate indicated that the molecule was transported more than 7 cm downstream (i.e. from root to leaves), and 1 cm upstream from the injection site. Upstream transport was likely the result of pure diffusive phenomena. The longer transport detected in the xylem when compared to phloem may be attributed to a more efficient deployment in its conduits, which also facilitated analysis conditions due to their larger diameter, and smaller background noise of green fluorescence. To quantify payloads transport, the fluorescence intensity was integrated (see FIG. 3E). The normalized intensity distribution evolves spatially and temporally (FIG. 3F). Notably, the dye was also transported along the radial system of the vasculature.

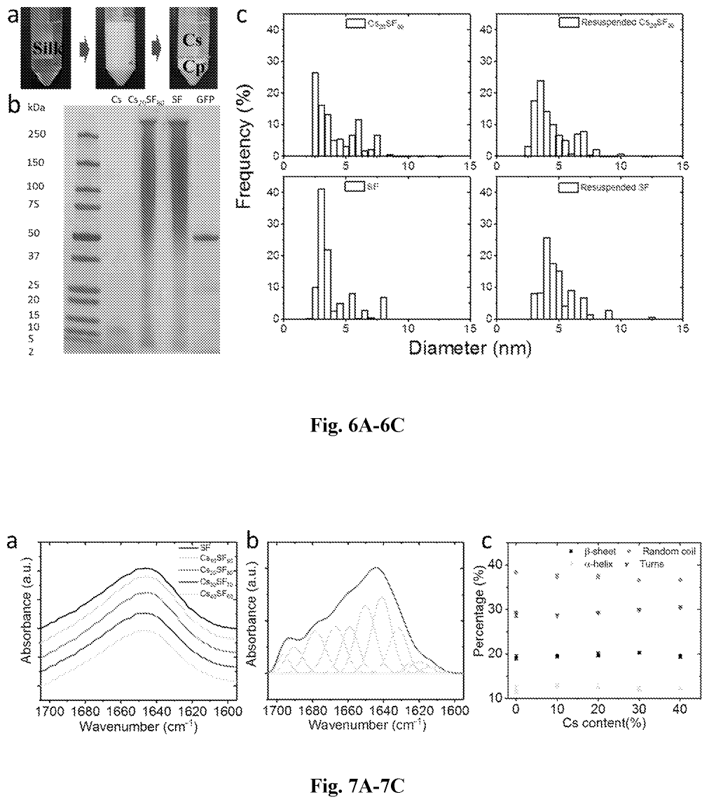

There are numerous examples of molecules, macromolecules, and bacteria that have been delivered in leaf tissue and roots to modify plants' genome, boost photosynthesis, and act as pesticide or fertilizer.^{[4]} Injection in the stem (or trunk) has also been performed to deliver antibiotics, pesticides, and nutrients.^{[22]} Here, to provide a proof of concept that the silk-based phytoinjectors of the invention can precisely orchestrate the deployment of different payloads in plant vasculature, a multi-reagents delivery system was designed that enables the well-known luciferin-luciferase bioluminescent reaction^{[7, 23]} in plant vasculature:

$$Luciferin + ATP + {O}_{2} \begin{matrix}{Mg}^{2 +} \\ \vec{Luciferase}\end{matrix} Oxyluciferin + AMP + PPi + {CO}_{2} + hv \qquad ( 1 )$$

where AMP is adenosine monophosphate, ATP is adenosine triphosphate, PPi is inorganic pyrophosphate and hv is light. A bioluminescent system was deployed in plant vasculature as a model for the complex biochemical interactions occurring during transport of hormones, signaling molecules, and peptides. Each phytoinjector is able to deliver a limited amount of payload—the total volume of xylem and phloem injector is 18.74±1.05 nl and 9.11±1.83 nl, respectively, and the tip volume (100 μm in length from the tip) is 0.135±0.010 nl and 0.030±0.005 nl, respectively. The phytoinjectors were applied in petiole vasculature near the leaflet to facilitate observation and imaging. At first, luciferin was deployed in the petiole's xylem while the other reagents were delivered by foliar infiltration to the leaf. The leaf tissues downstream the injection site showed luminescence (FIG. 4A), indicating the occurrence of the reaction, thus the delivery of luciferin and mobility of small molecules through the vasculature into ground tissue. Interestingly, no noticeable luminescence was observed from main veins, suggesting impermeability of vein structure to some reagents, likely luciferase due to its size. Luciferin and luciferase were then loaded to different phytoinjectors and injected to the same petiole (FIG. 4B), while the other reagents were infiltrated in the leaf ground tissue. Though faint, luminescence was detected in the vein of the leaf (FIG. 4B), indicating the delivery of multi reagents as well as a large protein via phytoinjectors.

Leveraging the polymorphic nature of silk materials, it was also possible to design water insoluble devices that reswell when exposed to sap fluid and can be removed post-injection. Such devices are here named phytosampler as they can be used to sample sap fluids. Since partial dissolution of the phytosampler is undesired, the phytosampler microneedle comprises greater than 90% pure silk fibroin as fabrication material. In embodiments, the phytosampler microneedle comprises greater than 100% pure silk fibroin.

The efficacy of the phytosampler was assessed by deploying it in the xylem downstream to a phytoinjector loaded with luciferin and Mg²⁺. Upon sampling, the phytosampler was exposed to the reaming reagent necessary for the bioluminescent reaction to occur. Generation of light indicated the successful sampling of luciferin and Mg′ from the xylem (FIG. 4C). The dislocation of phytoinjecor tip and luminescence spot in merged image is likely due to diffusion of luciferin into the solution drop of reagents and deformation of silk fibroin substrate when exposed to the reagents. Reswelling of the phytoinjectors and diffusion of metabolite and catabolite in silk phytosampler was modeled with a Lucas-Washburn equation^{[24]} (FIG. 4E) by investigating the diffusion of water and dyes like toluidine blue in the device (FIG. 4D and FIG. 4F), although poroelastic models^{[25]} could also be applied to take into account for the relaxation of the transient response of silk materials during reswelling.

To assess targeted delivery of live microorganisms into plant tissues, Agrobacterium tumefaciens with a pEAQ-HT vector containing gfp gene were loaded into Cs₂₀SF₈₀ phytoinjectors, using tobacco (Nicotiana benthamiana) as a model plant. A. tumefaciens has been widely used as a powerful gene transformation vehicle in plant genetic engineering to optimize the crop production of the desired products, such as drugs or proteins.^{[26]} A. tumefaciens-mediated genetic transformation can target: 1) developing tissues^{[27]}) inflorescences via floral dipping, or 3) leaves via foliar infiltration. Shoot apical meristems (SAMs), young growing leaves, and mature leaves were targeted. The phytoinjector dimensions were modified to optimize payload delivery via SAM injection and leaf injection (FIG. 1F, FIG. 15). At two weeks post-injection (when the SAM became a leaf), the leaves were harvested. Although all leaves exhibited GFP-induced fluorescence, the spatial distribution of GFP synthesis differed. Leaves derived from treated SAMs exhibited scattered GFP fluorescence across the leaf when excited with blue light (FIG. 5A(ii)). Using fluorescence microscopy, GFP expression was detected in multiple spots situated across the entire leaf (FIG. 5A(iii)), indicating successful gene transfer in mesophyll cells. The scattered distribution of these cells may result from cell divisions and subsequent growth of SAM cells. Since some (but not all) of the SAM cells that were directly in contact with A. tumefaciens (released from the Cs₂₀SF₈₀ phytoinjector) demonstrated gene transfer, it is hypothesized that GFP-expressing cells were separated from non-GFP-expressing cells during leaf growth. The young leaves grew in the two weeks post-injection, and GFP fluorescence in the form of lines or scattered spots situated was observed away from the injection site (FIG. 5B(iii)). This differs from what was observed in mature leaves, where GFP expression was limited to cells that are close to the injection site (FIG. 5C(iii)). The limited degree of gene transfer in mature leaves suggests that A. tumefaciens has little to no mobility upon release in the ground tissue. This is validated by foliar infiltration, where GFP expression in mesophyll cells is generally limited to the area directly accessible to A. tumefaciens. In growing young leaves, mesophyll cells can divide and grow, so GFP-expressing mesophyll cells form lines and scattered spots, depending on the geometrical growth of the leaf. Altogether, these results demonstrate that A. tumefaciens—mediated gene transfer to plant tissues can be achieved using Cs₂₀SF₈₀ phytoinjectors.

Microneedles have been previously reported for pain-free transdermal drug delivery and vaccination.^{[28]} As shown herein, the principles of biomaterial design were used to fabricate phytoinjector and phytosampler devices to deliver cargo molecules to plants and to investigate material transport phenomena in plant vasculature. Injection and silk degradation appeared to not compromise the functionality of both xylem and phloem and did not noticeably affect plant health, despite the formation of scar tissue around the injection site at day 14 post-injection (FIG. 15C). Immediate material degradation to nm-scale particles and the general bioinert nature of silk fibroin may, in fact, have resulted in a rapid recovery to physiological function upon flow disruption, with no evident adverse reaction to plant health at day 7 post-injection (FIG. 15C) and on sap flow (FIG. 3). Future studies are however necessary to investigate plant response to the injection, e.g. through studying Ca^{2+ [29]} and jasmonic acid signaling.^{[30]} The precise targeting of phloem here described may also open the door to future applications in systemic signaling molecules release in planta,^{[31]} which is currently not possible.

Accessing the phloem has in fact always been a technological challenge that is currently addressed using Pico gauge^{[32]} or by severing an aphid stylet during feeding.^{[33]} Precise injection in SAM also enabled the modification of plant genotype to induce expression in the current generation. The function of silk-based phytoinjectors was expanded herein to achieve analyte sampling from plant vasculature. Potential sampling applications of insoluble phytoinjectors include detection of early-stage phloem- and xylem-limited pathogens, natural plant response to environmental cues, and engineered plant response to user-defined cues. The design of plant-specific biomaterials to fabricate devices for drug delivery in planta opens new avenues to enhance plant resistance to biotic and abiotic stresses, provides new tools for diagnostics, and enables new opportunities in plant engineering.

### Experimental Section

Extraction of silk fibroin: An aqueous silk fibroin solution was prepared from Bombyx mori cocoons as described with modification.^{[34]} Briefly, dime size cocoon pieces were boiled for 45 minutes to remove sericin in 0.02 M sodium carbonate solution and dried overnight after thorough rinse in MilliQ water. The dried silk fibroin fibers were then dissolved in 9.3 M lithium bromide solution at 60° C. for 4 h, followed by dialysis against MilliQ water in a Slide-a-Lyzer dialysis cassette (MWCO 3500, Pierce, Rockford, Ill.) for 48 h. After centrifuge, the supernatant was obtained and stored at 4° C. prior to use. The final concentration of silk fibroin is roughly 7% w/v, determined by weighing the residual of 1 mL solution.

Cs preparation: Cs was prepared following the method described previously with modification.^{[13]} Alpha-chymotrypsin was added to aqueous silk fibroin solution by an enzyme to substrate weight ratio 1:100, followed by incubation at 37° C. for 24 h. The gel formed was then centrifuged at 4800×g for 30 minutes. The supernatant (Cs) was collected and kept at 80° C. for 20 minutes to denature alpha-chymotrypsin. The solution was centrifuged again, and the supernatant was stored at 4° C. prior to use. The concentration was determined by weighing dry residual.

Gel electrophoresis: The electrophoretic mobility of silk fibroin, Cs, and Cs₂₀SF₈₀ were determined using sodium dodecyl sulfate polyacrylamide gel electrophoresis (SDS-PAGE). 100 μg silk fibroin, 200 μg Cs, and 100 μg Cs₂₀SF₈₀ were reduced with 1 M dithiothreitol (DTT) and loaded into a precast 4-15% polyacrylamide gel (Bio-Rad Laboratories, Hercules, Calif.). The gel was run for 18 minutes at 200 V with a prestained recombinant protein mixture as reference (Bio-Rad Laboratories) and stained by Coomassie R-250 stain (Bio-Rad Laboratories).

Dynamic light scattering (DLS): Zeta Potential Analyzer (Brookhaven Instruments Corp., Holtsville, N.Y.) was used to measure the particle size in resuspended solution at a concentration of 2 mg ml⁻¹ dry material. Each measurement was 180 s and at least three measurements were carried out to confirm the reliability.

Circular dichroism (CD): CD experiments were conducted with a JASCO Model J-1500 Circular Dichroism Spectrometer (JASCO Co., Japan). Aqueous solutions were diluted to 0.01% w/v, loaded into a 1 mm path quartz cell (Starna Cells, Inc., Atascadero, Calif.), and scanned at 25° C. with a resolution of 0.5 nm and a 4 s accumulation time at the rate of 50 nm min⁻¹ from 250 nm to 185 nm wavelength. The results were averaged from three measurements.

Fourier Transform Infrared Spectroscopy (FTIR): IR measurements were carried out on a Spectrum 65 (PerkinElmer, Waltham, Mass.) equipped with an attenuated total reflection (ATR) generic UATR crystal, with a resolution of 4 cm⁻¹ and accumulation of 32 scans from 4000 and 650 cm⁻¹. Films were cast on PDMS, dried overnight, and kept in a desiccator for 24 h to remove surface water. Analysis was performed based on the Amide I region (1595-1705 cm⁻¹) by OriginPro 2017 software (OriginLab Corporation, Northampton, Mass.), following the previously described method.^{[15]}

Preservation of hydrogen peroxide and HRP: H₂O₂ can be enzymatically degraded by HRP, the product of which oxidizes 3,3′,5,5′-Tetramethylbenzidine (TMB) and generates a deep blue color. Upon addition of acid solution, the blue color turns to yellow that can be recorded absorbance at 450 nm. Briefly, for hydrogen peroxide preservation, H₂O₂ was added to CsSF blend solution, with a final H₂O₂ concentration 0.1% w/v and CsSF material concentration 6% w/v. Films were prepared by dropping 50 μl solution on PDMS and drying overnight in a fume hood. Each film was dissolved in 500 μl water for absorbance reading. 5 μl of the sample solution was mixed with 80 μl of TMB solution and incubated for 1 minute at room temperature before the addition of 100 μl 0.1 M sulfuric acid. Absorbance was detected at 450 nm with reference at 620 nm by a Tecan microplate reader (Tecan Group Ltd, Switzerland). HRP preservation shared a similar protocol with the modification where HRP was added to CsSF blend solution to prepare films.

Bacteria culture: Rhizobium tropici CIAT899 expressing bacterial GFP was obtained from Miguel Lara.^{[35]} R. tropici was cultured at 30° C. to OD600 of 1 following the instructions before use. GFP gene was cloned into pEAQ-HT vector and transformed into A. tumefaciens strain (LBA4404). Transformants were cultivated and selected at 30° C. for 24-36 h to OD600 of 1.5 in YM medium (0.4 g L⁻¹ yeast extract, 10 g L⁻¹ mannitol, 0.1 g L⁻¹ NaCl 0.2 g L⁻¹ MgSO₄.7H₂O, 0.5 g L⁻¹ K₂HPO₄.3H₂O, 15 g L⁻¹ agar, pH 7) supplemented with 50 μg mL⁻¹ rifampicin, 50 μg mL⁻¹ kanamycin, and 50 μg mL⁻¹ streptomycin.

Preservation of Agrobacterium tumefaciens: A. tumefaciens was cultured to OD600 1, centrifuged down at 3000×g for 30 minutes and resuspended by SF and Cs₂₀SF₈₀ to the same volume. Films were prepared by dropping 50 μl suspension on PDMS and drying overnight in a fume hood. The films were dissolved in 0.9% sterile NaCl solution and then spread on an agar plate for colony counting. A series of dilutions were prepared for better counting results.

Mechanical properties tests: Cs/silk fibroin solutions were cast on PDMS, dried overnight in a fume hood at room temperature, and cut into ribbons. Film tensile experiments were carried out on a Dynamic Mechanical Analysis (DMA) Q800 model (TA instruments, New Castle, Del.) with a strain rate of 0.5% min⁻¹ at room temperature. The static ultimate compression strength of phytoinjectors and puncture of tomato plants were conducted on an Instron-5943 (Instron, Norwood, Mass.) with a 10 N load cell at a loading speed of 10 mm min⁻¹. At least 7 samples were tested for each case. Nanoindentation measurements were performed on a Hysitron Tribolndenter with a nanoDMA transducer (Bruker, Billerica, Mass.). Samples were indented in load control mode with a peak force of 500 μN and a standard load-peak hold-unload function. Reduced modulus was calculated by fitting the unloading data (with upper and lower limits being 95% and 20%, respectively) using the Oliver-Pharr method. Each type of sample was prepared and indented in triplets to ensure good fabrication repeatability. For each sample, indentation was performed at a total of 49 points (7×7 grid with an increment of 20 μm in both directions) to ensure the statistical reliability of the modulus measurements.

Payloads release: Simulated sap was prepared according to the xylem exudate.^{[36]} Rhodamine 6 g and azoalbumin were added to SF and Cs₂₀SF₈₀ (6% w/v of dry materials) to get a final concentration of 0.1 mM and 2 mg ml⁻¹, respectively. R. tropici was centrifuged at 3000×g for 30 minutes and resuspended by SF and Cs₂₀SF₈₀ to get an OD600 of 1. The solutions were then cast on PDMS and dried overnight in a hood. The films were then cut into discs and attached to the bottom of a well of a 48 well plate, enabling only one side of the disc exposed to simulated sap. 1 ml of fresh simulated sap was added after the previous solution was collected for measurement. Released rhodamine 6 g and GFP-expressing R. tropici were monitored based on fluorescence intensity (excitation at 524 nm and 499 nm, emission at 550 nm and 520 nm). Released azoalbumin was monitored based on absorbance at 410 nm. At least three samples were tested for each case.

Master and negative mold fabrication: The aluminum master was fabricated by computer numerical control (CNC) machining with a 1/32″ flat end mill for rough milling, followed by a 1/64″ ball end mill for finishing. The templates were then chemically etched to the desired topologies based on application by aluminum etchant type A (Transene, Danvers, Mass.). To produce negative, Poly(dimethyl siloxane) (PDMS) (Sylgard 184, Dow-Corning, Midland, Mich.) was cast over Al master in a 60 mm petri dish, degassed, and finally incubated at 70° C. for 2 h.

Phytoinjector fabrication: The desired amount of payloads were mixed with Cs₂₀SF₈₀ solution and added to negative PDMS molds, followed by centrifuge at 1200×g for 15 minutes. Molds were then kept in a fume hood to dry at room temperature overnight.

Plant materials: Tobacco (Nicotiana benthamiana) and tomato (Solanum lycopersicum) plants were grown in pots in a plant chamber with ambient temperature 25° C. day/20° C. night and a 10 h photoperiod. Tobacco plants between 4-6 weeks old after germination are used for experiments, while tomato plants were used when they are 4-8 weeks old after germination.

Histology: Tomato plant tissues of interest were collected and kept in 10% formalin for 24 h, followed by immersion in 70% ethanol before processing by a Rapid Biopsy Processing on the Vacuum Infiltrating Tissue Processor for paraffin filling. 10 μm thick slices were prepared by a microtone and stained by Safranin 0 stain and Fast Green after deparaffinization.

TABLE 1
Power law fitting parameters of payloads release.

| | SF | | | Cs₂₀SF₈₀ | | |
|---|---|---|---|---|---|---|
| Material | k^{a)} | n | R² | k^{a)} | n | R² |
| Rhodamine 6G | 0.59 ± 0.04 | 0.93 ± 0.06 | 0.9823 | 27.54 ± 5.51 | 1.61 ± 0.07 | 0.9926 |
| Azoalbumin | 0.82 ± 0.34 | 1.13 ± 0.16 | 0.9395 | 8.63 ± 0.72 | 1.57 ± 0.04 | 0.9957 |
| R. tropici | 1.22 ± 0.17 | 1.12 ± 0.08 | 0.9850 | 15.52 ± 1.22 | 1.74 ± 0.04 | 0.9973 |

Data are mean ± s.d.
^{a)}The unit for time t is hour for paramater k.

Payload Release Profiles from SF and Cs₂₀SF₈₀

Payload release profiles in silk fibroin constructs have been studied extensively in controlled drug release applications,^{[28, 37]} with most studies indicating that diffusion, swelling, and proteolytic degradation are primary drivers in this process. As targeted plant tissues are not protease-rich, simulated sap was used to investigate payload release profile. Rhodamine 6G, azoalbumin, and GFP-expressing Rhizobium tropici CIAT 899 (GFP-CIAT 899) were used as representative models for small molecules, large proteins, and bacteria, and their release profiles in SF and Cs₂₀SF₈₀ were investigated. GFP-CIAT 899 was used in the release study in lieu of Agrobacterium as several attempts of staining Agrobacterium were inconclusive due to interaction between silk fibroin and the dyes used for live/dead assays. Silk fibroin and Cs₂₀SF₈₀ were found to have negligible effects on fluorescence and absorbance signal. The release profile of all three payloads for both silk fibroin and Cs₂₀SF₈₀ follow a power law (FIG. 12A) described by the semi-empirical model developed by Ritger and Peppas,^{[37]}

$${f}_{t} = \frac{{M}_{t}}{{M}_{∞}} = {kt}^{n} , \qquad ( S 1 )$$

which can be rewritten as lg (ƒₜ)=lg(k)+nlg(t), where ƒₜ is the fraction of released payload at time t, Mₜ is the amount of released payload over time t (unit: hour), M_{∞} is the amount of released payload at infinity time, (i.e., the total payloads loaded), k denotes the release velocity constant determined by the structural and geometric characteristic of the system, and n denotes the exponent of release indicating the release mechanism. Parameters for the power law were obtained by linear fitting, shown in Table S1. FIG. 12B depicts film surfaces of Cs₂₀SF₈₀ samples before release (silk fibroin samples have similar surfaces). Surface erosion is observed for all three releases from silk fibroin (FIG. 12C), while much faster payloads release and combination of surface and bulk erosion is observed for release from Cs₂₀SF₈₀ (FIG. 12D). Rhodamine 6G release from SF (n=0.93) is anomalous and dominated by both diffusion and swelling. Azoalbumin release (n=1.13) indicates a Super Case II release mechanism, possibly resulting from the secondary structure of azoalbumin (primarily a-helices) that lowers the interaction among silk fibroin chains and facilitates the disaggregation of swollen silk fibroin samples. GFP-CIAT 899 release is nearly identical to azoalbumin, but the sample surface shows protrusions, which display similar morphology to GFP-CIAT 899. All three payloads loaded into Cs₂₀SF₈₀ possessed a Super Case II release mechanism (n>1). This is likely due to the hydrophilicity of Cs, which dissolves easily in simulated sap and expedites the rate of sample degradation. These results show that Cs₂₀SF₈₀ allows for faster payload release profiles than SF, from small molecules, to large proteins, and to bacteria.

### Release and Transport Model in Xylem

The velocity of xylem sap flow is at the order of 10⁻³ m s⁻¹ although it varies a lot according to the condition of measured plants during the day^{[18]}. However, the velocity obtained here is at the order of 10⁻⁵ to 10⁻⁴ m s⁻¹, which may due to the influence of injection. This gives a Péclet number Pe=Lu/D˜10, where L is the diameter of xylem (˜10⁻⁴ m), u is the velocity of sap flow in xylem, and D denotes the diffusion coefficient of the payload delivered in xylem sap (10⁻¹⁰ m² s⁻¹). Thus both advection and diffusion should be taken into consideration in this scenario. The common form of the advection-diffusion equation for an impressible fluid without source and sink is

$$\frac{\partial c}{\partial t} = ∇ \cdot ( D ∇ c ) - v \cdot ∇ c \qquad ( S 2 )$$

Since the focus was on the longitudinal transport along xylem, Equation S1 can be simplified to one dimensional (1D) condition as

$$\frac{\partial c}{\partial t} = D \frac{{\partial }^{2} c}{\partial {x}^{2}} - u \frac{\partial c}{\partial x} \qquad ( S 3 )$$

The initial condition (IC) and boundary conditions (BCs) are as follow

IC: c(x, 0)=0
BCs: c(0, t)=c₀(t), c(∞, 0)=0.

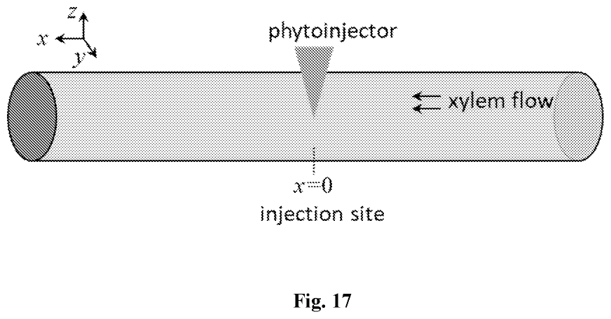

Once a phytoinjector is injected into xylem, the payload is released following the power law, contributing to the concentration change at x=0 at time t c₀(t). Mass conservation, i.e. payload released equals to that in the xylem, can be used to determine c₀(t). See FIG. 17 for a model.

To solve this problem, let

$$c ( x , t ) = Γ ( x , t ) {e}^{\frac{ux}{2 D} - \frac{{u}^{2} t}{4 D}} \qquad ( S 4 )$$

the Equation S3 can be rewritten as

$$\frac{\partial Γ}{\partial t} = D \frac{{\partial }^{2} Γ}{\partial {x}^{2}} \qquad ( S 5 )$$

$$IC : Γ ( x , 0 ) = 0$$

$$BCs : Γ ( 0 , t ) = f ( t ) = {c}_{0} ( t ) {e}^{\frac{{u}^{2} t}{4 D}} , Γ ( + ∞ , 0 ) = 0 .$$

Considering the Laplace transform of a function ƒ(x, t),

ƒ̅(x,s)=£[ƒ(x,t)]=∫₀^{+∞}e^{-st}ƒ(x,t)dt. (S6)

The Laplace transform of Equation S5 is

$$\frac{{d}^{2} \overline{Γ} ( x , s )}{{dx}^{2}} - \frac{s}{D} \overline{Γ} ( x , s ) = 0 \qquad ( S 7 )$$

subjecting to boundary conditions

$$\overline{f} ( s ) = \overline{Γ} ( 0 , s ) = {{\int }_{0}}^{+ ∞} {c}_{0} ( t ) {e}^{\frac{{u}^{2} t}{4 D} - st} dt , and \overline{Γ} ( + ∞ , s ) = 0.$$

The solution of Equation S7 is

$$\overline{Γ} ( x , s ) = \overline{f} ( s ) {e}^{- x \sqrt{\frac{s}{D}}} = ℒ [ f ( t ) * g ( x , t ) ] \qquad ( S 8 )$$

which can be considered as the Laplace transform of the convolution of two functions ƒ(t) and g(x, t), where

$$g ( x , t ) = {ℒ}^{- 1} [ {e}^{- \frac{x}{\sqrt{D}} \sqrt{s}} ] = \frac{x}{\sqrt{4 π {Dt}^{3}}} {e}^{- \frac{{x}^{2}}{4 Dt}} .$$

The inversion of Γ̅(x, s) gives

$$Γ ( x , t ) = {{\int }_{0}}^{t} f ( τ ) g ( x , t - τ ) d τ = \frac{x}{\sqrt{4 π D}} {{\int }_{0}}^{t} \frac{{c}_{0} ( τ )}{\sqrt{{( t - τ )}^{3}}} {e}^{\frac{{u}^{2} τ}{4 D} - \frac{{x}^{2}}{4 D ( t - τ )}} d τ . \qquad ( S 9 )$$

The concentration thus is

$$c ( x , t ) = \frac{x}{\sqrt{4 π D}} {e}^{\frac{ux}{2 D} - \frac{{u}^{2} t}{4 D}} {{\int }_{0}}^{t} \frac{{c}_{0} ( τ )}{\sqrt{{( t - τ )}^{3}}} {e}^{\frac{{u}^{2} τ}{4 D} - \frac{{x}^{2}}{4 D ( t - τ )}} d τ , ( x > 0 ) \qquad ( S 10 )$$

$$c ( x , t ) = \frac{- x}{\sqrt{4 π D}} {e}^{\frac{ux}{2 D} - \frac{{u}^{2} t}{4 D}} {{\int }_{0}}^{t} \frac{{c}_{0} ( τ )}{\sqrt{{( t - τ )}^{3}}} {e}^{\frac{{u}^{2} τ}{4 D} - \frac{{x}^{2}}{4 D ( t - τ )}} d τ , ( x < 0 ) \qquad ( S 11 )$$

Thus the concentration for the whole field is

$$c ( x , t ) = \frac{\vert x \vert}{\sqrt{4 π D}} {e}^{\frac{ux}{2 D} - \frac{{u}^{2} t}{4 D}} {{\int }_{0}}^{t} \frac{{c}_{0} ( τ )}{\sqrt{{( t - τ )}^{3}}} {e}^{\frac{{u}^{2} τ}{4 D} - \frac{{x}^{2}}{4 D ( t - τ )}} d τ . \qquad ( S 12 )$$

In addition, the concentration must meet mass conservation

Mₜ=M_{∞}ktⁿ=∫_{-∞}^{+∞}c(x,t)dx. (S13)

This integral equation determines boundary condition c(0, t)=c₀(t) and thus c(x, t). While it is hard to explicitly solve the integral equation, it can be solved numerically by Taylor series:

$${( \frac{\partial c}{\partial t} )}_{i}^{n} = \frac{{c}_{i}^{n + 1} - {c}_{i}^{n}}{Δ t} + O ( Δ t ) ,$$

$${( \frac{\partial c}{\partial x} )}_{i}^{n} = \frac{{c}_{i + 1}^{n} - {c}_{i - 1}^{n}}{2 Δ x} + O ( Δ {x}^{2} )$$

$${( \frac{{\partial }^{2} c}{\partial {x}^{2}} )}_{i}^{n} = \frac{{c}_{i + 1}^{n} - 2 {c}_{i}^{n} + {c}_{i - 1}^{n}}{Δ {x}^{2}} + O ( Δ {x}^{2} )$$

where n denotes time t and i is position x.

Equation S3 can be approximated as

$$\frac{{c}_{i}^{n + 1} - {c}_{i}^{n}}{Δ t} = D \frac{{c}_{i + 1}^{n} - 2 {c}_{i}^{n} + {c}_{i - 1}^{n}}{Δ {x}^{2}} - u \frac{{c}_{i + 1}^{n} - {c}_{i - 1}^{n}}{2 Δ x} + O ( Δ t , Δ {x}^{2} ) \qquad ( S 14 )$$

$$and$$

$${c}_{i}^{n + 1} = {c}_{i}^{n} - \frac{u Δ t}{2 Δ x} ( {c}_{i + 1}^{n} - {c}_{i - 1}^{n} ) + \frac{D Δ t}{Δ {x}^{2}} ( {c}_{i + 1}^{n} - 2 {c}_{i}^{n} + {c}_{i - 1}^{n} ) \qquad ( S 15 )$$

The code was written in MATLAB R2019a. Parameters to carry out the simulation used are D=4×10⁻¹⁰ m²/s, u=5×10⁻⁵ m/s, k=0.038 (for time unit minute), and n=1.61. The power law release describes well the first 60% payload release but not for 100%. Thus, the model well describes the release and transport in the first 5 minutes only. For longer time period, the payload loaded to other parts of the phytoinjector may also be released and contributes as payload source at the injection site, which invalidates the mass conservation assumption used here.

### Lucas-Washburn Model for Phytosampler

Reswelling of the phytoinjectors and diffusion of metabolite and catabolite in silk phytosampler was modeled with a Lucas-Washburn equation.^{[24]} The fitting was carried out in MATLAB R2019a Curve Fitting Toolbox on collected data of penetration depth of water frontier in a phytosampler over time.

The fitting equation is

H=36.42\sqrt{t−54.32}, (S16)

where H is the penetration depth, t is time (unit second). The adjusted R²=0.9932. The time t₀=54.32 s may attribute to the cone shape of the phytosampler, which does not match the 1D case for Lucas-Washburn model.

### MATLAB Code for Payloads Release

function release
%This function is used to solve the release of payloads from
%phytoinjector and transport in xylem and phloem.
%The model is 1D advection-diffusion equation.
%Constants
%D=7.0*10\hat{ }(−10); %Diffusion coefficient of Mg2+ ion in water
%D=4.0*10\hat{ }(−10); %Diffusion coefficient of R6G/5(6)-Carboxyfluorescein diacetate in
water
%D=6.1*10\hat{ }(−11); %Diffusion coefficient of albumin in water

| | |
|---|---|
| %u=10\hat{ }(−3); | %Velocity of sap in xylem |
| %u=10\hat{ }(−4); | %Velocity of sap in phloem |

D=4.0*10\hat{ }(−10);
u=5*10\hat{ }(−5);

| | |
|---|---|
| k=0.038; | %M=Minf*k*t\hat{ }(nn), M/Minf<=60, tmax is calculated |

nn=1.61;
Minf=1;
tmax=round(60*(0.6/k)\hat{ }(1/nn));%Total time,unit second
%tmax=300;

| | |
|---|---|
| dt=0.001; | %Time step, |

tN=tmax/dt;

| | |
|---|---|
| L=0.1; | %2N+1 is the number of points along x L=0.1m |

N=10000;
dx=L/(2*N);
%Matrice
c_tn=zeros(1,2*N+1); %t=n*dt Concentration of payloads at each point
c_tn1=zeros(1,2*N+1); %t=(n+1)*dt Concentration of payloads at each point
c_x0=zeros(1,tN+1); %c_x0(t), Concentration at x=0, c(N+1). t=0,c0(1)=0

| | |
|---|---|
| cinf=0; | %Concentration at infinite, c(1)=c(2*N+1)=0 |

```
x=−L/2:dx:L/2;
t_output=[60 180 300]; %used to determine when to write c(x,t), −L/2<=x<=L/2
ct=zeros(length(t_output),2*N+1);
ij=0;
for n=0:tN−1
  t=(n+1)*dt;
  cn_tem=c_tn;
  m_err=1e−6;
  aa_lower=0;aa_upper=1;aa=1;
  while abs(m_err)>1e−8
    if aa>0
      [aa,aa_upper,aa_lower]=increase(m_err,aa,aa_upper,aa_lower);
    %else
      % aa_lower=−1;aa_upper=0;aa=−1;
      % [aa,aa_upper,aa_lower]=increase(m_err,aa,aa_upper,aa_lower);
    end
    %Initialization
    M_tn=Minf*k*(dt/60)\hat{ }nn*((n+1)\hat{ }nn−(n)\hat{ }nn); % material released at tn
    c_x0(n+2)=c_x0(n+1)+aa*M_tn/dx; %c0(x=0,t)
    cn_tem(N+1)=c_x0(n+2);
    c_tn1(1)=cinf;  %BCs x=−L/2
    c_tn1(2*N+1)=cinf; %x=L/2
    for i=2:N*2
      c_tn1(i)=cn_tem(i)−...
          u*dt/(2*dx)*(cn_tem(i+1)−cn_tem(i−1))+...
          D*dt/(dx)\hat{ }2*(cn_tem(i+1)−2*cn_tem(i)+cn_tem(i−1));
      if c_tn1(i)<0
        c_tn1(i)=0;
      end
    end
    %material released error during n to n+1 dt period
    m_err=sum((c_tn1−c_tn))*dx−M_tn;
  end
  c_x0(n+2)=c_tn1(N+1); %c(x=0,t=t)
  c_tn=c_tn1;
  %used to determine when to write c(x,t), at every 0.1*tmax
  if ismember((n+1)*dt,t_output)
    ij=ij+1;
    ct(ij,:)=c_tn;
  end
end
t=0:dt:tmax;
fileID = fopen(‘concentration vs time.txt’,‘w’);
fprintf(fileID,‘%10s %12s %12s %12s\r\n’,‘x’,‘t0’,‘t1’,‘t2’);
fprintf(fileID,‘%10.8f %12.8f %12.8f %12.8f\r\n’,[x;ct]);
fclose(fileID);
fileID2 = fopen(‘c_x0 vs time’,‘w’);
fprintf(fileID2,‘%10s %12s\r\n’,‘time(s)’,‘c_x0’);
fprintf(fileID2,‘%10.8f %12.8f\r\n’,[t;c_x0]);
fclose(fileID2);
figure
ax1=subplot(2,1,1);
grid on
plot(ax1,t,c_x0)
title(‘concentration at x=0 vs. time’)
xlabel(ax1,‘Time(s)’)
ylabel(ax1,‘Concentration’)
ax2=subplot(2,1,2);
grid on
plot(ax2,1000*x,ct(:,:))
title(‘concentration distribution at different time’)
xlabel(ax2,‘x(mm)’)
ylabel(ax2,‘Concentration’)
end
function [aa,aa_upper,aa_lower]=increase(m_err,aa,aa_upper,aa_lower)
  if m_err>0
    aa_upper=aa;
  else
    aa_lower=aa;
  end
  aa=(aa_lower+aa_upper)/2;
end.
```

### REFERENCES

[1] Food Security Information Network. Global Report on food Crises 2018, 2018.
[2] S. Takeda, M. Matsuoka, Nat. Rev. Genet. 2008, 9, 444; S. S. Zaidi, M. S. Mukhtar, S. Mansoor, Trends Biotechnol. 2018, 36, 898; R. Gibbers, V I. Adarnchuk, Science 2010, 327, 828.
[3] W. Elmer, J. C. White, Annu. Rev. Phytopathol. 2018, 56, 111.
[4] J. Fischer, S. J. Beckers, D. Yiamsawas, E. Thines, K. Landfester, F. R. Wurm, Adv. Sci. 2019, 1802315; N. Mitter, E. A. Worrall, K. E. Robinson, P. Li, R. G. Jain, C. Taochy, S. J. Fletcher, B. J. Carroll, G. M. Lu, Z. P. Xu. Nat. Plants 2017, 3, 16207.
[5] R. Paul, A. C. Saville, J. C. Hansel, Y. Ye, C. Ball, A. Williams, X. Chang, G. Chen, Z. Gu, J. B. Ristaino, Q. Wei, ACS Nano 2019, 13, 6540.
[6] J. P. Giraldo, M. P. Landry, S. M. Faltermeier, T. P. McNicholas, N. M. Iverson, A. A. Boghossian, N. F. Reuel, A. J. Hamer, F. Sen, J. A. Brew, M. S. Strano, Nat. Mater. 2014, 13, 400; S. Y. Kwak, T. T. S. Lew C. J. Sweeney, V. B. Koman, M. H. Wong, K. Bohmert-Tatarev, K. D. Snell, J. S. Seo, N. H. Chua, M. S. Strano, Nat. Nanotechnol. 2019, 14, 447.
[7] S. Y. Kwak, J. P. Giraldo, M. H. Wong, V B. Koman, T. T. S. Lew, J. Ell, M. C. Weidman, R, M. Sinclair, M. P. Landry, W. A. Tisdale, M. S. Strano, Nano Lett. 2017, 17, 7951,
[8] N. Wang, E. A. Pierson, J. C. Setubal, J. Xu, J. G. Levy, Y. Zhang, J. Li, L. T. Rangel, J. Martins, Jr., Annu. Rev. Phytopathol. 2017, 55, 451; J. Bové, M. Gamier, Plant Sci. 2002, 163, 1083.
[9] C. Holland, K. Numata, J. Rnjak-Kovacina, F. P. Seib, Adv. Healthe. Mater. 2019, 8.
[10] Z. Zhou, S. Zhang, Y. Cao, B. Marelli, X. Xia, T. H. Tao, Adv. Mater 2018, 30, 1706983.
[11] B. Marelli, M. Brenckle, D. L. Kaplan, F. G. Omenetto, Sci. Rep. 2016, 6, 25263.
[12] M. C. White, A. M. Decker, R. L. Chaney, Plast Physiol. 1981, 67, 292.
[13] B. Marelli, C. E. Ghezzi, A. Alessandrino, J. E. Barralet, G. Freddi, S. N. Nazhat, Biomaterials 2012, 33, 102.
[14] B. Ranjbar, P. Gill, Chem. Biol. Drug Des. 2009, 74, 101.
[15] X. Hu, D. Kaplan, P. Cebe, Macromolecules 2006, 39, 6161.
[16] N. Smirnoff, D. Arnaud, New Phytol. 2019, 221, 1197.
[17] H. J. Jin, J. Park, V Karageorgiou, U. J. Kim, R. Valluzzi, P. Cebe, D. L. Kaplan, Adv. Funct. Mater 2005, 15, 1241.
[18] K. H. Jensen, K. Berg-Sçrensen, H. Bruus, N. M. Holbrook, J. Liesche, A. Schulz, M. A. Zwieniecki, T. Bohr, Rev. Mod. Phys. 2016, 88.
[19] B. Choat, A. R. Cobb, S. Jansen, New Phytol. 2008, 177, 608.
[20] D. L, Mullendore, C. W, Windt, H. Van As, M. Knoblauch, Plant Cell 2010, 22, 579.
[21] J. A. Savage, M. A. Zwieniecki, M. Holbrook, Plant Physiol. 2013, 163, 1409.
[22] C. Berger, F. Laurent, Crop Prot. 2019, 124.
[23] T. Nakatsu, S. Ichiyama, J. Hiratake, A. Saldanha, N. Kobashi, K. Sakata, H. Kato, Nature 2006, 440, 372.
[24] E. W. Washburn, Phys. Rev. 1921, 17, 273.
[25] E. Detournay, A. H.-D. Cheng, Analysis and design methods, Elsevier, Amsterdam, 1993.
[26] P. N, Desai, N, Shrivastava, H, Padh, Biotechnol. Adv. 2010, 28, 427.
[27] P. Baskaran, Soós, E. Balázs, J. Van Stader, S. Afr. J. Bot. 2016, 103, 210.
[28] K. Tsioris, W. K. Raja, E. M. Pritchard, B. Panilaitis, D. L. Kaplan; F. G. Omenetto, Adv. Funct. Mater. 2012, 22, 330; E. Larraneta, R. E. Lutton, A. D. Woolfson, R. F. Donnelly, Mater: Sci. Eng. R Rep. 2016, 104, 1; J. A. Stinson, W. K. Raja, S. Lee, H. B, Kim, I. Diwan S. Tutunjian, B. Panilaitis, F. G. Omenetto, S. Tzipori, D. L. Kaplan, ACS Biomater. Sci. Eng. 2017, 3, 360.
[29] M. Toyota, D. Spencer, S. Sawai-Toyota, W. Jiaqi, T. Zhang, A. J. Koo, G. A. Howe, S. Gilroy, Science 2018, 361, 1112.
[30] C. Wasternack, I. Stenzel, B. Hause, G. Hause, C. Kutter, H. Maucher, J. Neumerkel, I. Feussner, O. Miersch, J. Plant Physiol. 2006, 163, 297.
[31] M. Heil, J. Ton, Trends Plant Sci. 2008, 13, 264; A. Santner, M. Estelle, Nature 2009, 459, 1071; Y. Ohkubo, M. Tanaka, R. Tabata, M. Ogawa-Ohnishi, Y. Matsubayashi, Nat. Plants 2017, 3, 17029; B. K. Ham, W. J. Lucas, Annu. Rev. Plant Biol. 2017, 68, 173.
[32] J. Knoblauch, D. L. Mullendore, K. H. Jensen, M. Knoblauch, Plant Physiol. 2014, 166, 1271.
[33] D. B. Fisher, C. E. Cash-Clark, Plant Physiol. 2000, 123, 125; J. P, Wright, D. B. Fisher, Plant Physiol. 1980, 65, 1131
[34] D. N. Rockwood, R. C. Preda, T. Yucel, X. Wang, M. L. Lovett, D. L. Kaplan, Nat. Protoc. 2011, 6, 1612.
[35] K. Nanjareddy, L. Blanco, M. K. Arthikala, X. A. Affantrange, F. Sanchez, M. Lara, J. Integr. Plant Biol. 2014, 56, 281.
[36] M. C. White, A. M. Decker, R. L. Chaney, Plant Physiology 1981, 67, 292; J. Bialczyk, Z. Lechowsk, J. Plant Nutr. 1995, 18, 2005.
[37] Q. Lu, B. Zhang, M. Li, B. Zuo, D. L. Kaplan, Y. Huang, H. Zhu, Biomacromolecules 2011, 12, 1080; D. J. Hines, D. L. Kaplan, Biomacromolecules 2011, 12, 804.

1. A composition comprising a mixture of (a) polypeptides derived from silk fibroin (Cs) that have a higher hydrophilic content than pure silk fibroin; and (b) pure silk fibroin (SF).

2. The composition of claim 1, wherein the dry weight ratio of Cs:SF is between 10:90 and 30:70.

3.-4. (canceled)

5. The composition of claim 1, wherein the dry weight percentage of Cs is at least 21%.

6. (canceled)

7. A method for manufacturing a silk fibroin material having a higher hydrophilic content than pure silk fibroin comprising: (a) contacting pure SF with a proteolytic enzyme under conditions sufficient to degrade SF proteins thereby forming hydrophilic polypeptides (Cs); and (b) combining the Cs with SF to form a mixture having a higher hydrophilic content than pure SF.

8. The method of claim 7, wherein the dry weight ratio of Cs:SF is between 10:90 and 30:70.

9.-10. (canceled)

11. The method of claim 7, wherein the dry weight percentage of Cs is at least 21%.

12. (canceled)

13. A method for delivering a payload to a locus in plant tissue comprising (a) providing a ratio of Cs:SF material configured as a microneedle device having a higher hydrophilic content than pure SF; (b) loading the microneedle device with the payload; and (c) contacting the plant tissue with the microneedle device under conditions sufficient to allow the payload to enter the locus.

14. The method of claim 13, wherein the dry weight ratio of Cs:SF in the microneedle is between 10:90 and 30:70.

15.-16. (canceled)

17. The method of claim 13, wherein the dry weight percentage of Cs is at least 21%.

18. (canceled)

19. A microneedle comprising (a) the composition of claim 1, wherein the microneedle comprises a base and at least one penetrating tip, or (b) a base and a penetrating tip, wherein the penetrating tip is capable of sampling fluid in the xylem or phloem of a plant.

20. The microneedle of claim 19, wherein the dry weight ratio of Cs:SF is between 10:90 and 30:70.

21.-22. (canceled)

23. The microneedle of claim 19, wherein the dry weight percentage of Cs is at least 21%.

24. (canceled)

25. The microneedle of claim 19, wherein the penetrating tip length is suitable for penetration of the microneedle to the xylem of phloem of a plant.

26. The microneedle of claim 19, wherein the penetrating tip diameter is capable of penetrating the xylem or phloem of a plant without disrupting the flow of material in the xylem or phloem.

27. The microneedle of claim 26, wherein the tip diameter is smaller than 35 μm.

28. The microneedle of claim 19, comprising at least one active agent selected from the group consisting of proteins, peptides, antigens, immunogens, vaccines, antibodies or portions thereof, antibody-like molecules, enzymes, nucleic acids, siRNA, shRNA, aptamers, viruses, bacteria, small molecules, cells, hormones, antibiotics, therapeutic agents, diagnostic agents, and any combinations thereof.

29.-30. (canceled)

31. A diagnostic kit for diagnosing the presence of a pathogen in the xylem or phloem of a plant comprising a microneedle of claim 19.

32. (canceled)
